(19) United States
(12) Patent Application Publication
(10) Pub. No.: US 2014/0364407 A1
(43) Pub. Date: Dec. 11, 2014

(54) MEDICAL OR THERAPEUTIC APPLICATION OF A COMPOSITE MATERIAL OF RUTHENIUM WITH A NITROSYL LIGAND

(71) Applicant: CENTRE NATIONAL DE LA RECHERCHE, Paris (FR)

(72) Inventors: Isabelle Malfant, Goyrans (FR); Benoit Cormary, Montgiscard (FR)

(73) Assignee: CENTRE NATIONAL DE LA RECHERCHE SCIENTIFIQUE - CNRS, Paris Cedex 16 (FR)

(21) Appl. No.: 14/362,741

(22) PCT Filed: Dec. 5, 2012
(86) PCT No.: PCT/FR2012/052815

§ 371 Date: Jun. 4, 2014

(30) Foreign Application Priority Data
Dec. 6, 2011 (FR) 1161223

(51) Int. Cl. C07F 15/00; A61K 31/555; A61K 41/00

(52) U.S. Cl. CPC C07F 15/0053; A61K 41/0042; A61K 31/555
USPC 514/188

(57) ABSTRACT

A medical or therapeutic application of a composite material of ruthenium with a nitrosyl ligand. Application for a medical or therapeutic treatment of a composite material of ruthenium with a nitrosyl ligand irradiated in a spectral range capable of releasing the nitrosyl ligand, i.e., the ultraviolet or visible or near-infrared spectral range.

The invention relates to the application, for a medical or therapeutic treatment, of a composite material of ruthenium with a nitrosyl ligand.

The therapeutic applications of the NO molecule regarding a number of physiological processes, such as neurotransmission and the destruction of cancer cells, are already known. Thus, Robert F. Furchgott, Ferid Murad and Louis J. Ignarro were awarded the Nobel prize for Medicine and Physiology in 1988 for having established the decisive role of NO in cardiovascular physiology. In addition, it is known that, while NO has an anti-apoptotic role, it also has a pro-apoptotic role.

The invention aims to make it possible to take greater advantage of the role of NO in the medical or therapeutic field.

Document WO 2010/081977 describes a sol-gel process for producing a photochromic composite material, such a photochromic composite material thus prepared, and the application of such a material as a high-quality, in particular high-capacity, optical memory medium.

The invention is based on the surprising observation that such a material or a derived material could also be used for a medical or therapeutic treatment based on the role of NO.

The subject of the invention is therefore a composite material of ruthenium with a nitrosyl ligand able to release the nitrosyl ligand when it is irradiated in a spectral range, for use in a medical or therapeutic treatment of the human or animal body.

As described in greater detail below, said composite material of ruthenium with a nitrosyl ligand comprises a photochromic complex of ruthenium with a nitrosyl ligand, of formula:

[Ru(NO)X(py-R)₄]Y₂, [Ru(NO)X(py-RR′)₄]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br, BF₄ and PF₆,
R, R′, R″ are chosen from the family comprising hydrogen, alkyl groups, alcohol groups, aldehyde groups, ketone groups, ester groups, ether groups, amine groups, amide groups and halogenated groups,
or of formula [RuCl(NO)(py)₄](PF₆)₂.1/2H₂O.

According to one embodiment, the composite material of ruthenium with a nitrosyl ligand is able to release the nitrosyl ligand locally and in a photocontrolled manner when it is irradiated in a spectral range, such as the ultraviolet and visible spectral range or that of near-infrared.

According to one embodiment, the composite material of ruthenium with a nitrosyl ligand is in solution.

According to one possible embodiment, the composite material of ruthenium with a nitrosyl ligand is irradiated in the ultraviolet and visible spectral range, i.e. in a wavelength range of between 300 nm and 700 nm.

According to another possible embodiment, the composite material of ruthenium with a nitrosyl ligand is irradiated in the near-infrared spectral range, i.e. in a wavelength range of between 700 nm and 1100 nm.

According to a first possible embodiment, the composite material of ruthenium with a nitrosyl ligand is prepared by means of a sol-gel process with:
an alkoxysilane as precursor,
a silica matrix, and
a photochromic complex of ruthenium with a nitrosyl ligand, a complex of formula:

[Ru(NO)X(py-R)4]Y2, [Ru(NO)X(py-RR′)4]Y2, [Ru(NO)X(py-RR′R″)4]Y2 or [Ru(NO)X(py)4]Y2 wherein:
py denotes pyridine and Ru denotes ruthenium,
X is chosen from the family comprising Cl, Br and OH,
Y is chosen from the family comprising Cl, Br, BF4 and PF6,
R, R′, R″ are chosen from the family comprising hydrogen, alkyl groups, alcohol groups, aldehyde groups, ketone groups, ester groups, ether groups, amine groups, amide groups and halogenated groups,
or of formula [RuCl(NO)(py)₄](PF₆)₂.1/2H₂O.

According to one embodiment, the alkoxysilane is selected from the group comprising tetramethoxyorthosilane—TMOS-, vinyltriethoxysilane—VTES-, tetraethoxyorthosilane—TEOS-, or alkoxides M(OR)ₓ, wherein M is a metal and R is an alkyl group.

According to a second possible embodiment, the composite material of ruthenium with a nitrosyl ligand is prepared with a biocompatible matrix, in particular of a polysaccharide such as starch (glucan) or agar-agar (galactan), and selected as photochromic complex of ruthenium with a nitrosyl ligand is a complex of formula [Ru(NO)X(py-R)₄]Y₂, [Ru(NO)X(py-RR′)₄]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br and BF₄,
R, R′, R″ are chosen from the family comprising hydrogen, alkyl groups, alcohol groups, aldehyde groups, ketone groups, ester groups, ether groups, amine groups, amide groups and halogenated groups.

The term “biocompatible” is intended to mean which can be received by the human body without damage. Matrices of polysaccharide such as starch (glucan) or agar-agar (galactan) have the advantage of also being biodegradable.

A material such as that used for the application as medical or therapeutic treatment is a composite material of ruthenium with a nitrosyl ligand irradiated in a spectral range able to release the nitrosyl ligand.

In the case of a preparation by means of a process of sol-gel type, according to the first embodiment envisioned, reference is expressly made to document WO 2010/081977, already mentioned, which describes such a process. The sol-gel method comprises the following successive steps:
hydrolysis,
condensation or, denoted otherwise, polymerization or gelling,
drying.

In the case of a second embodiment envisioned with a biocompatible matrix, the steps of dissolving the biocompatible matrix, of dispersing the photochromic complex, of placing in a container, and of drying, wherein the temperature and the time are selected, are carried out such that, in the matrix prepared, depending on the shape of the container, the photochromic complex of ruthenium with a nitrosyl ligand, in the crystalline state and in the form of nanoparticles, is inserted into the nanopores of the matrix.

As indicated, according to the two embodiments envisioned, a silica matrix or a biocompatible matrix, in particular of starch or of agar-agar, is selected as matrix.

In combination, and in the case of a silica matrix, selected as photochromic complex of ruthenium with a nitrosyl ligand is a complex of formula:

[Ru(NO)X(py-R)a]Y₂, [Ru(NO)X(py-RR′)a]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br, BF₄ and PF₆,
R, R′, R″ are chosen from the family comprising hydrogen, alkyl groups, alcohol groups, aldehyde groups, ketone groups, ester groups, ether groups, amine groups, amide groups and halogenated groups,

or of formula [RuCl(NO)(py)₄](PF₆)₂.1/2H₂O.

In the case of a biocompatible matrix, selected as photochromic complex of ruthenium with a nitrosyl ligand is a complex of formula [Ru(NO)X(py-R)a]Y₂, [Ru(NO)X(py-RR′)a]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br and BF₄,
R, R′, R″ are chosen from the family comprising hydrogen, alkyl groups, alcohol groups, aldehyde groups, ketone groups, ester groups, ether groups, amine groups, amide groups and halogenated groups.

The alkyl groups comprise, for example, linear or branched alkyls having 1 to 4 carbon atoms, such as methyl, ethyl, n-propyl, isopropyl or n-butyl.

The alcohol groups comprise, for example, —OH or alkyl chains having 1 to 4 carbon atoms substituted with one or more —OH groups.

The aldehyde groups comprise, for example, —COH or alkyl chains having 1 to 4 carbon atoms substituted with one or more —COH groups.

The ester groups comprise, for example, —COR1, wherein R₁ is an alkyl having 1 to 4 carbon atoms.

The ether groups comprise, for example, —OR₁, wherein R₁ is an alkyl having 1 to 4 carbon atoms.

The amine groups comprise, for example, —NH₂, —NHR₁ or —NR₁R₂, wherein R₁ and R₂ are independently alkyls having 1 to 4 carbon atoms.

The amide groups comprise, for example, —CONH₂, —CONHR₁ or —CONR₁R₂, wherein R₁ and R₂ are independently alkyls having 1 to 4 carbon atoms.

The halogenated groups comprise, for example, chlorine, bromine, iodine or fluorine or alkyl chains having 1 to 4 carbon atoms substituted with one or more halogen atoms, such as chlorine, bromine, iodine or fluorine.

Pyridine py, monosubstituted pyridine py-R, disubstituted pyridine py-RR′ and trisubstituted pyridine py-RR′R″ can be illustrated by the following diagrammatic structures:

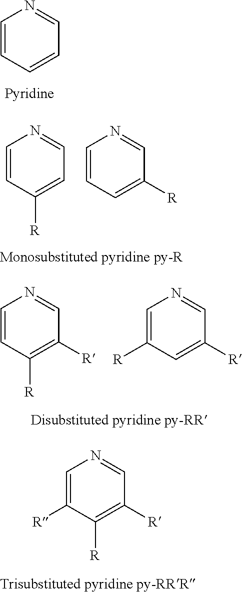

In one implementation of the process according to the first embodiment and the sol-gel process, tetramethoxyorthosilane—TMOS-is used as precursor. In other possible implementations, vinyltriethoxysilane—VTES-, or even tetraethoxyorthosilane—TEOS-, or more generally alkoxides M(OR)x, wherein M is a metal, such as aluminum or titanium, x is an integer which can range up to 4, and R is an alkyl group (having 1 to 4 carbon atoms), which are functionally equivalent, are used.

As indicated, it has been demonstrated, surprisingly, that a composite material of ruthenium with a nitrosyl ligand as previously described, once irradiated in a spectral range able to release the nitrosyl ligand, can have applications for a medical or therapeutic treatment based on the role of NO.

The medical or therapeutic treatment comprises, for example:
the treatment of cardiovascular diseases, such as infarction,
the treatment of cancers, such as skin cancers,
the treatment of ocular diseases, such as macular degeneration.

More especially, the composite material of ruthenium with a nitrosyl ligand is irradiated in a spectral range able to release the nitrosyl ligand locally and in a photocontrolled manner, for example in the ultraviolet or visible spectral range, i.e. in a wavelength range of between 300 nm and 700 nm.

According to another possible embodiment, the composite material of ruthenium with a nitrosyl ligand is irradiated in the near-infrared spectral range, i.e. in a wavelength range of between 700 nm and 1100 nm.

The medical or therapeutic treatment may be carried out by dynamic phototherapy. It is thus possible either to administer the composite material of ruthenium with a nitrosyl ligand orally, or to apply it percutaneously, and then the area to be treated is irradiated with an appropriate radiation so as to locally release the nitrosyl ligand.

Furthermore, according to one possible embodiment, the composite material of ruthenium with a nitrosyl ligand used for the medical or therapeutic application under consideration is in solution.

The subject of the invention is also a pharmaceutical composition comprising the composite material of ruthenium with a nitrosyl ligand as defined above, and a pharmaceutically acceptable excipient. The pharmaceutical composition may be in various forms, for example in the form of a tablet or of a gel capsule or else of a patch.

The dose administered will, of course, depend on the therapeutic application envisioned and on the patient to be treated. The pharmaceutical composition administered will comprise a sufficient amount of composite material of ruthenium with a nitrosyl ligand to make it possible to locally release a therapeutically effective amount of NO. The amount of NO locally released can be controlled by the irradiation wavelength and time. The amount of NO released can be measured, for example, by means of an NO electrode, of the Grieff test or else of the Co TPP test, and the appropriate wavelength can thus be adjusted so that the material releases NO.

1-8. (canceled)

9. A method for treating a disease based on the action of nitric oxide in a patient, said method comprising:
(1) administering to said patient a therapeutically effective amount of a composite material of ruthenium with a nitrosyl ligand able to release the nitrosyl ligand when it is irradiated in an ultraviolet or visible spectral range,
(2) irradiating an area of said patient to be treated with an ultraviolet or visible radiation, wherein said material comprising a photochromic complex of ruthenium with a nitrosyl ligand, is of formula:
[Ru(NO)X(py-R) ₄]Y₂, [Ru(NO X(py-RR′)₄]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br, BF₄ and PF₆,
R, R′, R″ are chosen from the family comprising hydrogen, alkyl groups, alcohol groups, aldehyde groups, ketone groups, ester groups, ether groups, amine groups, amide groups and halogenated groups,
or of formula [RuCl(NO)(py)₄](PF₆)₂.1/2H₂O.

10. A method for treating a disease based on the action of nitric oxide in a patient, said method comprising:
(1) administering to said patient a therapeutically effective amount of a composite material of ruthenium with a nitrosyl ligand able to release the nitrosyl ligand when it is irradiated in a near-infrared spectral range,
(2) irradiating an area of said patient to be treated with a near-infrared radiation, wherein said material comprising a photochromic complex of ruthenium with a nitrosyl ligand, is of formula:
[Ru(NO)X(py-R)₄]Y₂, [Ru(NO)X(py-RR′)₄]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br, BF₄ and PF₆,
R, R′, R″ are chosen from the family comprising hydrogen, alkyl groups, alcohol groups, aldehyde groups, ketone groups, ester groups, ether groups, amine groups, amide groups and halogenated groups,
or of formula [RuCl(NO)(py)₄](PF₆)₂.1/2H₂O.

11. The method as claimed in claim 9, able to be irradiated in an ultraviolet or visible spectral range and to release the nitrosyl ligand locally in a photocontrolled manner.

12. The method as claimed in claim 9, prepared by means of a sol-gel process with:
an alkoxysilane as precursor,
a silica matrix, and
a photochromic complex of ruthenium with a nitrosyl ligand, a complex of formula:
[Ru(NO)X(py-R)₄]Y₂, [Ru(NO)X(py-RR′)₄]Y₂[Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br, BF₄ and PF₆,
R, R′, R″ are chosen from the family comprising hydrogen, alkyl groups, alcohol groups, aldehyde groups, ketone groups, ester groups, ether groups, amine groups, amide groups and halogenated groups,
or of formula [RuCl(NO) (py)₄](PF₆)₂.1/2H₂O.

13. The method as claimed in claim 9, wherein the alkoxysilane is selected from the group consisting of tetramethoxyorthosilane—TMOS-, vinyltriethoxysilane—VTES-, tetraethoxyorthosilane—TEOS-, and alkoxides M(OR)x, wherein M is a metal and R is an alkyl group.

14. The method as claimed in claim 9, prepared with a biocompatible matrix, in particular of starch or of agar-agar, and, as photochromic complex of ruthenium with a nitrosyl ligand, a complex of formula:
[Ru(NO)X(py-R) ₄]Y₂, [Ru(NO)X(py-RR′)₄]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br and BF₄,
R, R′, R″ are chosen from the family comprising hydrogen, alkyl groups, alcohol groups, aldehyde groups, ketone groups, ester groups, ether groups, amine groups, amide groups and halogenated groups.

15. A pharmaceutical composition comprising the composite material of ruthenium with a nitrosyl ligand as defined in claim 9 and a pharmaceutically acceptable excipient.

16. The pharmaceutical composition as claimed in claim 15 in the form of a tablet, a gel capsule or a patch.

17. The method as claimed in claim 9, wherein said disease is chosen from cardiovascular diseases, cancers or ocular diseases.

18. The method as claimed in claim 9, wherein said composite material of ruthenium with a nitrosyl ligand able to release the nitrosyl ligand when it is irradiated in an ultraviolet or visible spectral range is administered to said patient orally or percutaneously.

19. The method as claimed in claim 10, able to be irradiated in a near-infrared spectral range and to release the nitrosyl ligand locally in a photocontrolled manner.

20. The method as claimed in claim 10, prepared by means of a sol-gel process with:
an alkoxysilane as precursor,
a silica matrix, and
a photochromic complex of ruthenium with a nitrosyl ligand, a complex of formula:
[Ru(NO)X(py-R)₄]Y₂, [Ru(NO)X(py-RR′)₄]Y₂[Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br, BF₄ and PF₆,
R, R′, R″ are chosen from the family comprising hydrogen, alkyl groups, alcohol groups, aldehyde groups, ketone groups, ester groups, ether groups, amine groups, amide groups and halogenated groups,
or of formula [RuCl(NO) (py)₄](PF₆)₂.1/2H₂O.

21. The method as claimed in claim 10, wherein the alkoxysilane is selected from the group consisting of tetramethoxyorthosilane—TMOS-, vinyltriethoxysilane—VTES-, tetraethoxyorthosilane—TEOS- and alkoxides M(OR)x, wherein M is a metal and R is an alkyl group.

22. The method as claimed in claim 10, prepared with a biocompatible matrix, in particular of starch or of agar-agar, and, as photochromic complex of ruthenium with a nitrosyl ligand, a complex of formula:
[Ru(NO)X(py-R)₄]Y₂, [Ru(NO)X(py-RR′)₄]Y₂, [Ru(NO)X(py-RR′R″)₄]Y₂ or [Ru(NO)X(py)₄]Y₂ wherein:
py denotes pyridine and Ru denotes ruthenium,
X is chosen from the family comprising Cl, Br and OH,
Y is chosen from the family comprising Cl, Br and BF₄,
R, R′, R″ are chosen from the family comprising hydrogen, alkyl groups, alcohol groups, aldehyde groups, ketone groups, ester groups, ether groups, amine groups, amide groups and halogenated groups.

23. A pharmaceutical composition comprising the composite material of ruthenium with a nitrosyl ligand as defined in claim 10 and a pharmaceutically acceptable excipient.

24. The pharmaceutical composition as claimed in claim 21 in the form of a tablet, a gel capsule or a patch.

25. The method as claimed in claim 10, wherein said disease is chosen from cardiovascular diseases, cancers or ocular diseases.

26. The method as claimed in claim 10, wherein said composite material of ruthenium with a nitrosyl ligand able to release the nitrosyl ligand when it is irradiated in a near-infrared spectral range is administered to said patient orally or percutaneously.
